(19) United States
(12) Patent Application Publication
(10) Pub. No.: US 2019/0183456 A1
(43) Pub. Date: Jun. 20, 2019

(54) INFERRING HEART FIBER GEOMETRY FROM ULTRASOUND IMAGING

(71) Applicant: THE ROYAL INSTITUTE FOR THE ADVANCEMENT OF LEARNING/MCGILL UNIVERSITY, Montreal (CA)

(72) Inventors: Peter SAVADJIEV, Montreal (CA); Kaleem SIDDIQI, Westmount (CA)

(21) Appl. No.: 16/327,906

(22) PCT Filed: Jun. 23, 2017
(86) PCT No.: PCT/CA2017/050771

§ 371 Date: Feb. 25, 2019

Related U.S. Application Data
(60) Provisional application No. 62/379,918, filed on Aug. 26, 2016.

(51) Int. Cl. A61B 8/08; G16H 30/20

(52) U.S. Cl. CPC A61B 8/0883; A61B 8/5207; G16H 30/20; A61B 8/5246; A61B 8/5269; A61B 8/5223

(57) ABSTRACT

The present disclosure provides methods and systems for determining heart fiber geometry from ultrasound images. Ultrasound images of a heart model are generated. Then, a probability of accuracy of the model ultrasound images is determined based at least in part on one or more ultrasound image of an actual heart. When the probability of accuracy is above a given threshold, the heart model is applied to the ultrasound images of the actual heart in order to determine heart fiber geometry for the actual heart.

### CROSS-REFERENCE TO RELATED APPLICATIONS

The present application claims the benefit of U.S. Provisional Patent Application No. 62/379,918 filed on Aug. 26, 2016, the contents of which are hereby incorporated in their entirety by reference.

### TECHNICAL FIELD

The present disclosure relates generally to medical imaging, and more specifically to ultrasound-based heart imaging.

### BACKGROUND OF THE ART

In simple terms, a heart is a muscle composed of cardiac muscle cells. Cardiac muscle cells are elongated cells which can contract to cause the heart to pump blood to flow through the heart and to other parts of the body. Cardiac muscle cells take the form of fibrous elements, the substrate of which are myofibers. Myofibers channel electrical activation of cardiac muscles during normal heart beat cycles, and allow cardiac muscles to pump blood efficiently. The position and orientation of myofibers can be indicative of damage or disease in a heart; it may thus be useful to determine myofiber geometry for a heart as a diagnostic tool for detecting, amongst others, cardiac remodeling following injury to the heart. Determining myofiber geometry can also be used for monitoring medication-based therapies, as well as for planning surgical procedures for treatment of, for instance, arrhythmias, tissue resection, or for pacemaker placement.

Traditional research in myofiber geometry has focused on MRI-based (magnetic-resonance imaging) techniques. Certain types of MRI, such as diffusion MRI (dMRI), have allowed for heart tissues to be imaged with sufficient precision to allow for orientation mapping of myofibers. However, performing dMRI in vivo in clinics is challenging: most modern dMRI techniques are only applicable either to excised hearts, or to in vivo experiments in heavy research contexts, because dMRI techniques are very sensitive to motion.

In contrast, ultrasound-based heart imaging is widely used in cardiology, as ultrasound imaging is versatile, portable, easy to use, and inexpensive. However, technological challenges, including high noise levels and ambiguous intensity readings, have prevented ultrasound imaging from being applied to living patients for performing myofiber geometry assessments for a living heart.

As such, there is a need for ultrasound-based heart myofiber imaging techniques which can be applied in vivo.

### SUMMARY

The present disclosure provides methods and systems for determining heart fiber geometry from ultrasound images. Ultrasound images of a heart model are generated. Then, a probability of accuracy of the model ultrasound images is determined based at least in part on one or more ultrasound image of an actual heart. When the probability of accuracy is above a given threshold, the heart model is applied to the ultrasound images of the actual heart in order to determine heart fiber geometry for the actual heart.

In accordance with a broad aspect, there is provided a method for determining heart fiber geometry for a heart, comprising: generating ultrasound images of a heart model; determining a probability of accuracy of the ultrasound images of the heart model based at least in part on at least one ultrasound image of an actual heart; and when the probability of accuracy is above a given threshold, applying the heart model to the at least one ultrasound image to determine heart fiber geometry of the actual heart.

In some embodiments, determining the probability of accuracy of the ultrasound images of the heart model comprises: determining a similarity level indicative of a similarity between the ultrasound images of the heart model and the at least one ultrasound image of the actual heart; determining a cohesion level indicative of a similarity between adjacent pixels of the ultrasound images of the heart model; and computing the probability of accuracy of the ultrasound images of the heart model based on the similarity level and the cohesion level.

In some embodiments, determining a similarity level comprises applying a sum-of-squared differences algorithm.

In some embodiments, determining a cohesion level comprises applying an average angular difference algorithm.

In some embodiments, determining a probability of accuracy comprises applying a Bayesian interference belief propagation algorithm.

In some embodiments, the method further comprises, when the probability of accuracy is below the given threshold: creating new ultrasound images based on a new heart model; and determining the probability of accuracy for the new ultrasound images.

In some embodiments, generating ultrasound images of a heart model comprises generating the ultrasound images of a mathematical heart model validated against an image dataset acquired using diffusion magnetic resonance imaging.

In some embodiments, generating ultrasound images of a heart model comprises generating the ultrasound images of the heart model from at least two different incoming ultrasound beam directions.

In some embodiments, the method further comprises obtaining the at least one ultrasound image of the actual heart.

In some embodiments, obtaining the at least one ultrasound image of the actual heart comprises obtaining the at least one ultrasound image data set in vivo.

According to another broad aspect, there is provided a system for determining heart fiber geometry for a heart, comprising a processing unit; and a non-transitory memory communicatively coupled to the processing unit and comprising computer-readable program instructions executable by the processing unit. The program instructions are executable for: generating ultrasound images of a heart model; determining a probability of accuracy of the ultrasound images of the heart model based at least in part on at least one ultrasound image of an actual heart; and when the probability of accuracy is above a given threshold, applying the heart model to the at least one ultrasound image to determine heart fiber geometry of the actual heart.

In some embodiments, determining the probability of accuracy of the ultrasound images of the heart model comprises: determining a similarity level indicative of a similarity between the ultrasound images of the heart model and the at least one ultrasound image of the actual heart; determining a cohesion level indicative of a similarity between adjacent pixels of the ultrasound images of the heart model; and computing the probability of accuracy of the ultrasound images of the heart model based on the similarity level and the cohesion level.

In some embodiments, determining a similarity level comprises applying a sum-of-squared differences algorithm.

In some embodiments, determining a cohesion level comprises applying an average angular difference algorithm.

In some embodiments, determining a probability of accuracy comprises applying a Bayesian interference belief propagation algorithm.

In some embodiments, the computer-readable program instructions are further executable for, when the probability of accuracy is below the given threshold: creating new ultrasound images based on a new heart model; and determining the probability of accuracy for the new ultrasound images.

In some embodiments, generating ultrasound images of a heart model comprises generating the ultrasound images of a mathematical heart model validated against an image dataset acquired using diffusion magnetic resonance imaging.

In some embodiments, generating ultrasound images of a heart model comprises generating the ultrasound images of the heart model from at least two different incoming ultrasound beam directions.

In some embodiments, the computer-readable program instructions are further executable for obtaining the at least one ultrasound image of the actual heart.

In some embodiments, obtaining the at least one ultrasound image of the actual heart comprises obtaining the at least one ultrasound image data set in vivo.

Features of the systems, devices, and methods described herein may be used in various combinations, and may also be used for the system and computer-readable storage medium in various combinations.

### BRIEF DESCRIPTION OF THE DRAWINGS

Further features and advantages of embodiments described herein may become apparent from the following detailed description, taken in combination with the appended drawings, in which:

FIG. 1 is a diagram of an example human heart pre-and-post imaging.

FIG. 2 is a flowchart of an example method for determining heart fiber geometry.

FIG. 3 is a flowchart of an example Bayesian inference process.

FIG. 4 is a graphical representation of an example two-layer Markov random field.

FIG. 5 is a flowchart of an example implementation of a step of the method of FIG. 2.

FIG. 6 is a schematic diagram of an example computing system for implementing the method of FIG. 2 in accordance with an embodiment.

FIG. 7 is a block diagram of an example implementation of an heart fiber geometry determination system.

FIGS. 8A-B are example ultrasound images of a heart model.

FIG. 9A is an example ground truth heart myofiber geometry.

FIG. 9B is an example heart myofiber geometry as determined by an embodiment of the method of FIG. 2.

FIG. 10 is an example two-dimensional slice of an echocardiography acquisition from an example patient.

FIG. 11 is the example two-dimensional slice of FIG. 10 with an example highlighted region.

FIGS. 12A and 12B are respectively wide and zoomed-in views of a fiber orientation field as determined by an embodiment of the method of FIG. 2.

It will be noted that throughout the appended drawings, like features are identified by like reference numerals.

### DETAILED DESCRIPTION

Ultrasound imaging is performed by directing high frequency sound waves at a portion of the body and detecting the interaction of the sound waves with the body. Ultrasound imaging may be based on transmission rates, reflection rates, attenuation rates, and the like. Ultrasound imaging can be used to obtain images of a heart, even in vivo (i.e., performed or taking place on a living organism).

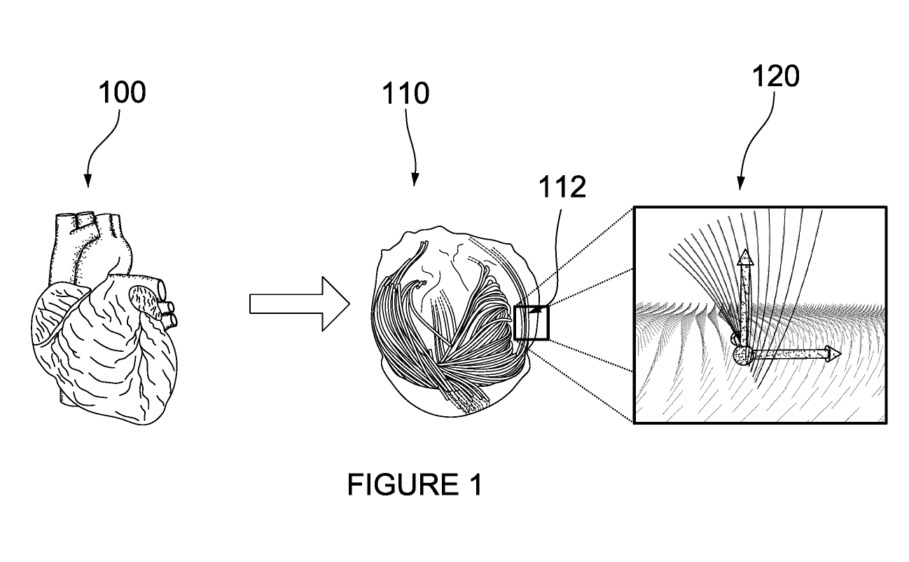

With reference to FIG. 1, an example human heart 100 is shown. The heart 100 may be modeled as a model heart 110, and may be made up of a plurality of myofibers 112. Ultrasound imaging, such as ultrasound backscatter (USBS) imaging, is used to provide information indicative of an angle between an incoming ultrasound angle and myofiber orientation. Myofiber orientation can be illustrated as a generalized helicoid model 120. In order to use USBS imaging to determine heart fiber geometry for a heart, including for in vivo hearts, ultrasound images of a heart model are generated. The ultrasound images of the heart model are based on a virtual model of a heart having certain myofiber geometries. Then, a probability of accuracy of the model ultrasound images is determined based, at least in part, on one or more ultrasound images of an actual heart. The ultrasound images of the actual heart can be acquired, for example, from an ultrasound probe. When the probability of accuracy is above a given threshold, the heart model is determined to be an accurate representation of the heart and is applied to the ultrasound images of the actual heart in order to determine heart fiber geometry of the heart.

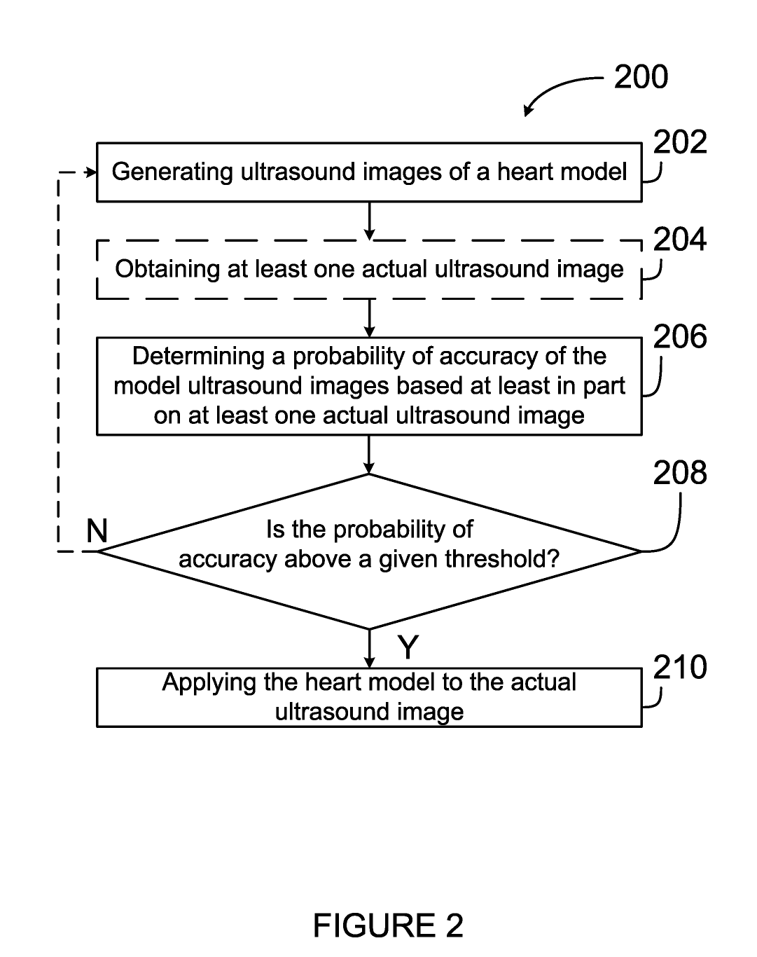

With reference to FIG. 2, a method 200 for determining heart fiber geometry for a heart, such as the heart 100, is illustrated. At step 202, ultrasound images of a heart model are generated. The ultrasound images of the model are generated, for example, by simulating the one or more interactions between incident ultrasound beams and a model of a heart. The heart model may be based on any suitable representation of a heart, for example any suitable mathematical heart model. In some embodiments, the mathematical heart model is validated against an image dataset of one or more hearts acquired using diffusion magnetic resonance imaging (dMRI), or any other heart model. The interaction between the heart model and incident ultrasound beams can be simulated using any suitable software. In some embodiments, the simulation software used is the COLE ultrasound simulation software.

The simulation of the interaction between the heart model and incident ultrasound beams is used to produce ultrasound images of the heart model, referred to herein as model ultrasound images. The model ultrasound images may be brightness-mode (B-mode) ultrasound images, amplitude-mode (A-mode) ultrasound images, motion-mode (M-mode) ultrasound images, or any other suitable type of ultrasound image. For example, the model ultrasound images are B-mode ultrasound images, where the intensity of the images at each location, or pixel, is indicative of the amount of USBS from the tissue at that location. Alternatively, ultrasound elastography images can be acquired, for example by shear wave imaging, or by other suitable methods. In some embodiments, the model ultrasound images are a plurality of sets of model ultrasound images acquired via a respective plurality of simulated ultrasound beams. In some such embodiments, some of the plurality of simulated ultrasound beams are incident from different angles from one another, with respect to the heart model. Thus, different sets of model ultrasound images may be captured by simulated ultrasound beams incident from different directions. In some embodiments, at least two model ultrasound image sets are acquired. In other embodiments, a single model ultrasound image set is acquired. The model ultrasound images may also be composite images.

Optionally, the method 200 includes obtaining at least one ultrasound image of an actual heart at step 204, referred to herein as an actual ultrasound image. The actual ultrasound image may be captured via an ultrasound sensor or probe, for example from a patient, from a cadaver, from a sample, and the like, or may be acquired from a database, described in greater detail hereinbelow. The actual ultrasound image is of the heart 100, and may be acquired using any suitable ultrasound imaging technique. For example, the actual ultrasound image is a B-mode USBS image. In some embodiments, an ultrasound imaging technique analogous to that used to generate the model ultrasound images at step 202 is used at step 204.

In embodiments where the model ultrasound images are the plurality of sets of model ultrasound images acquired via a respective plurality of simulated ultrasound beams, an equivalent plurality of sets of at least one actual ultrasound image are acquired via an equivalent and respective plurality of actual ultrasound beams. For example, if model ultrasound images are generated for two different incident angles at step 202, then actual ultrasound images for two different angles are acquired at step 204. The actual ultrasound image may also be a composite image.

At step 206, a probability of accuracy of the model ultrasound images is determined based, at least in part, on the at least one actual ultrasound image. In some embodiments, the probability of accuracy is determined via a Bayesian inference approach, described in greater detail hereinbelow. In other embodiments, other types of machine learning are used, which include both supervised and non-supervised machine learning. For example, any one or more of deep learning, artificial neural networks, sparse dictionary learning, and the like, are used. The probability of accuracy of the model ultrasound images may be expressed in any suitable way, including as a ratio, as a percentage of success, as a raw probability, and the like. In some embodiments, the probability of accuracy of the model ultrasound images is stored in a database, for example in conjunction with the model ultrasound images.

At step 208, a decision is made regarding the probability of accuracy of the model ultrasound images. If the probability of accuracy is below a certain threshold, the method 200 optionally returns to step 202 to generate new model ultrasound images. The new model ultrasound images may be based on a new heart model, a modified heart model, or may be based on the same heart model but acquired from one or more different angles or using one or more different techniques. If the probability of accuracy is above the threshold, the method 200 advances to step 210.

In some embodiments, if the probability of accuracy is at or near the threshold, or within a predetermined tolerance of the threshold, the method 200 may advance to an additional validation step (not illustrated) in which the heart model used to generate the model ultrasound images at step 202 is further validated. The additional validation step may involve generating additional model ultrasound images, for example from different angles or of different resolution, and determining the probability of accuracy of the additional model ultrasound images. Alternatively, the additional validation step may involve using additional actual ultrasound images to reassess the probability of accuracy of the model ultrasound images. Still other additional validation steps are considered.

At step 210, the heart model is output. The heart model may be output to a program or other software, for example a visualization software. Alternatively, or in addition, the heart model may be stored in a database or other storage medium. Alternatively still, the model may have a flag associated therewith, set to indicate the accuracy of the model. Still other ways of outputting the heart model are considered.

As discussed hereinabove, determining the probability of accuracy of the model ultrasound images may be accomplished via a Bayesian inference approach. Given input data about a system, and a mathematical model for that system, Bayesian inference can be used to infer the most likely set of model parameters to describe the input data. In general terms, this process is built around three concepts: a prior, a likelihood, and a posterior. The prior encodes prior knowledge about the model and the model parameters. Given a specific set of values for the model parameters, the likelihood encodes the confidence in model outputs based on the specific values for the model parameters. The posterior encodes the confidence in the model parameters given the observed data. Computing the posterior is used to determine a best-fit model.

Bayes' theorem describes a probability of an event occurring based on conditions that may or may not be related to the event. More formally, Bayes' theorem states that the probability of an event A occurring given the occurrence of an event B is:

$$P ( A \vert B ) = \frac{P ( B \vert A ) P ( A )}{P ( B )}$$

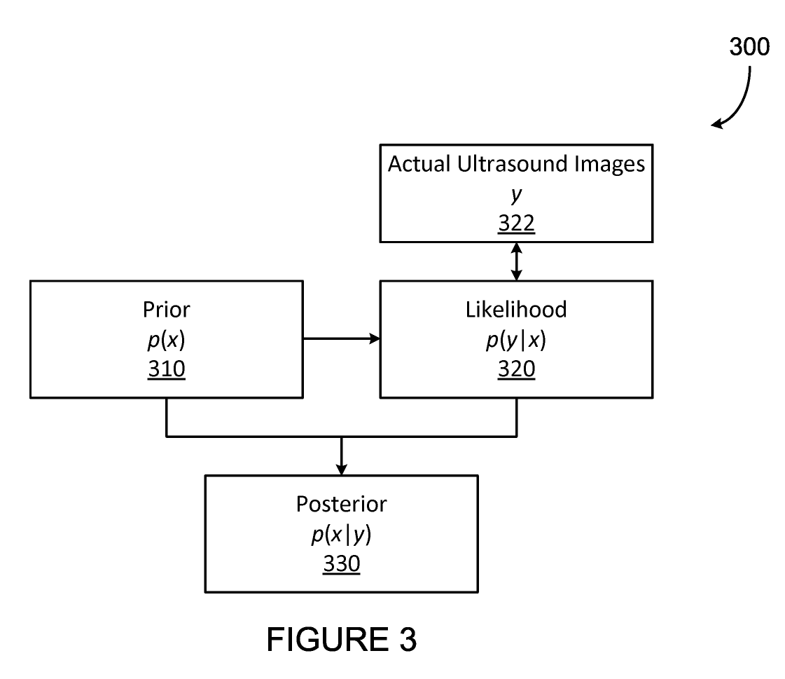

With reference to FIG. 3, in the Bayesian inference framework, prior knowledge about the model and its parameters is expressed as a probability distribution on the parameters, called the prior 310, p(x), where x represents a vector, or set, of model parameters. The likelihood 320 is representative of newly observed information about the model parameters, and is proportional to the conditional probability distribution of the observed data y given the model parameters x. Put differently, the likelihood 320 is proportional to the conditional probability that a set of observed data y is true if the model parameters x are accurate, written as p(y|x).

The likelihood 320, p(y|x), and the prior 310, p(x), are then combined to obtain a probability distribution for the model parameters given the data, the posterior 330, written as p(x|y). From Bayes' theorem, the posterior 330 can be defined as the product of the prior 310 and the likelihood 320, divided by the probability distribution of the observed data p(y), written as:

$$p ( x \vert y ) = \frac{p ( y \vert x ) p ( x )}{p ( y )}$$

Since p(y) is assumed to be constant, it can be disregarded, and the equation for the posterior 330 is written as

p(x|y) ∝ p(y|x) p(x)

As it pertains to heart fiber geometries, x is the model ultrasound images and y is the actual ultrasound image. Thus, the probability that the model ultrasound images are accurate given the actual ultrasound image, p(x|y), is proportional to the probability of the actual ultrasound image being accurate given the model ultrasound images, p(y|x), times the probability of the model ultrasound images being accurate, p(x). The prior 310 can be based on established mathematical models for the three-dimensional structure of heart fiber geometry. The mathematical models may be based, for example, on dMRI scanning of one or more hearts. In some embodiments, the model may be a generalized helicoid model

Many different algorithms may be used to implement the above-described Bayesian inference approach. One such algorithm is the belief propagation (BP) algorithm. The BP algorithm is an iterative algorithm which performs Bayesian inference on graphs: data structures composed of nodes connected with edges. Graphs may be of particular applicability to computer vision applications, as each pixel in an image can be assigned an associated node, and edges can be used to link adjacent pixels' nodes to represent the spatial relationship between pixels.

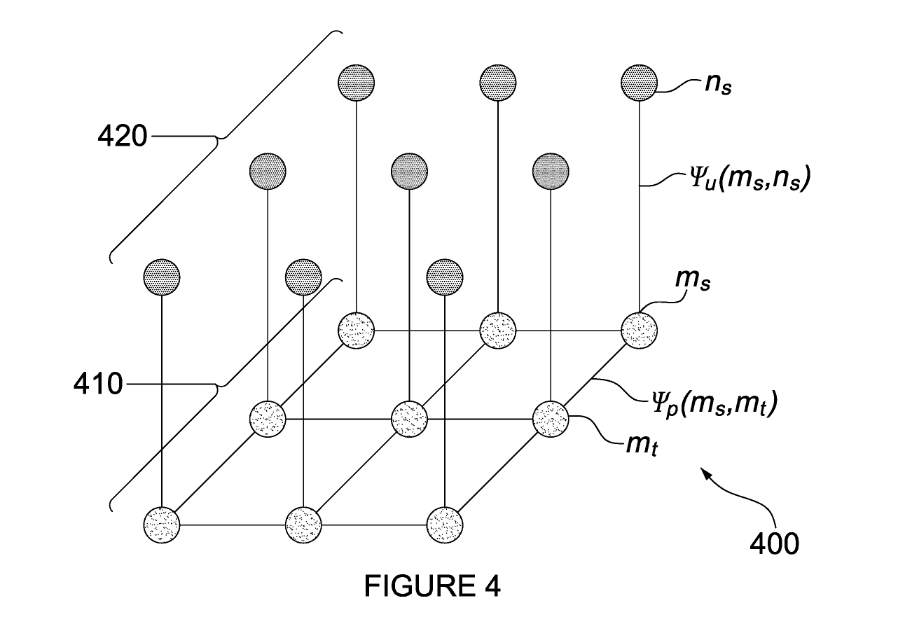

With reference to FIG. 4, in some embodiments the graph used with the BP algorithm is a Markov Random Field (MRF) 400. The MRF 400 is composed of a first layer of hidden nodes 410 and a second layer of observable nodes 420. The hidden nodes 410 are linked by edges to both adjacent hidden nodes 410 and vertices to one associated observable node 420. The observable nodes 420 are only linked by vertices to one of the hidden nodes 410. The hidden nodes 410 correspond to estimated values for each pixel in the actual ultrasound image, i.e. the model ultrasound images, which are based on parameters of the heart model, and the observable nodes 420 correspond to pixels of the actual ultrasound image.

For the MRF 400, let m denote the random variables corresponding to all of the hidden nodes 420 in the model, m={m₁, m₂, . . . mᵢ}, i.e. the model ultrasound images, and let {n₁, n₂, . . . nᵢ} denote the corresponding set of observed nodes 410, i.e., the actual ultrasound images. If the MRF 400 has a set of vertices V and a set of edges E, the probability of the estimated values in the hidden nodes 420 being correct, p(m), can be expressed as

$$p ( m ) ∝ {\prod }_{s ∈ V}^{ } {ψ}_{u} ( {m}_{s} , {n}_{s} ) {\prod }_{( s , t ) ∈ E}^{ } {ψ}_{p} ( {m}_{s} , {m}_{t} )$$

The BP algorithm will seek to maximize the probability p(m) to find a most probable configuration for the variables of the hidden nodes 410 in the MRF 400. Because the MRF 400 includes many loops (i.e., closed paths formed by the edges of four adjacent vertices), a particular variant of BP, called loopy-BP, may be used to find a maximum for p(m), or an approximation thereof. The equation for p(m) is proportional to two terms: ψᵤ(mₛ, nₛ), which is referred to as the unary potential of the hidden node mₛ, and ψₚ(mₛ, mₜ), which is referred to as the pairwise potential of hidden nodes mₛ and mₜ. This equation mirrors the equation for p(x|y) discussed hereinabove.

The unary potential ψᵤ(mₛ, nₛ) is a function of both a hidden node mₛ and an observable node nₛ. The unary potential is a measure of similarity between the model ultrasound image and the actual ultrasound image: in other words, it corresponds to the likelihood p(y|x). The pairwise potential ψₚ(mₛ, mₜ), on the other hand, is a function of two adjacent hidden nodes mₛ and mₜ. The pairwise potential is a measure of smoothness or compatibility between the hidden nodes mₛ and mₜ, or more generally of their cohesion: in other words, it corresponds to the prior p(x).

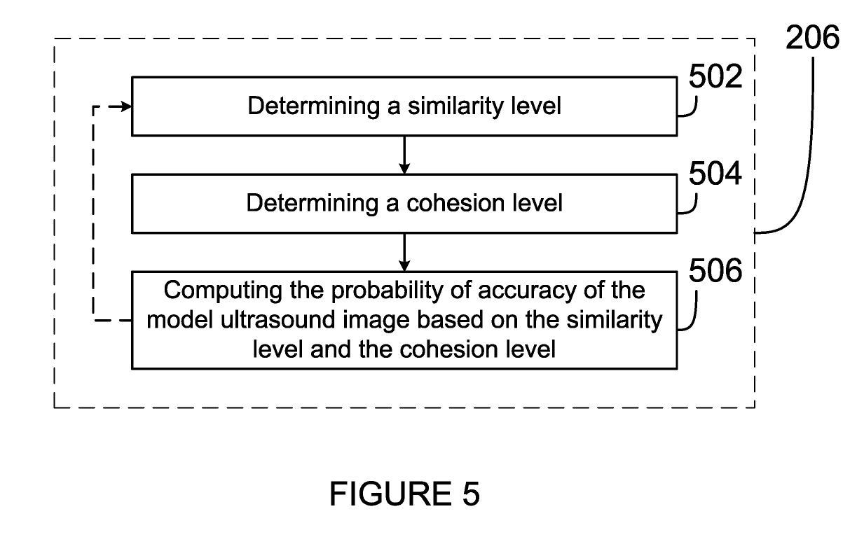

Thus, with reference to FIG. 5, in some embodiments step 206 of the method 200 comprises a series of steps 502, 504, 506. At step 502, the unary potential ψᵤ(mₛ, nₛ) is obtained by determining a similarity level indicative of similarity between the model ultrasound images and the at least one actual ultrasound image. The similarity level may be any suitable value or representation of any existing similarity between the model ultrasound images and the actual ultrasound image. For example, the similarity level is a number indicative of a number of matching pixels between the model ultrasound images and the actual ultrasound image. Alternatively, the similarity level is a score representative of the overall agreement of the pixels of the model ultrasound images to the pixels of the actual ultrasound image. In some embodiments, the similarity level is determined by applying a sum-of-squared differences algorithm. In other embodiments, other algorithms are used, for example, normalized correlation, mutual information, and the like.

At step 504, the pairwise potential ψₚ(mₛ, mₜ) is obtained by determining a cohesion level indicative of similarity between adjacent pixels of each of the model ultrasound images. Specifically, for each of the model ultrasound images, each pixel is compared to each of the pixels adjacent thereto. The cohesion level is then indicative of the similarity determined by these comparisons. The cohesion level may be any suitable value or representation of any existing similarity between adjacent pixels of the model ultrasound images, and may be similar to the similarity level of step 502. In some embodiments, the cohesion level is determined by applying an average angular difference algorithm. In other embodiments, other algorithms are used, for example algorithms based on quaternions and Euler angles, orientation distribution functions, probabilistic methods based on distributions such as the von Mises-Fisher distribution, and the like.

At step 506, the probability of accuracy of the model ultrasound images is computed based on the similarity level of step 502 and the cohesion level of step 504. In particular the probability of accuracy can be computed by taking the product of the unary potential ψᵤ(mₛ, nₛ) and the pairwise potential ψₚ(mₛ, mₜ). The BP algorithm can be applied iteratively to refine the random variables assigned to the hidden nodes 420 and recalculating the unary potential ψᵤ(mₛ, nₛ) and the pairwise potential ψₚ(mₛ, mₜ). At convergence, repeating steps 502, 504, 506 may result in a maximum estimate of the random variables of the hidden nodes 420, and the probability of accuracy can then be calculated.

In some embodiments, the method 200, and the steps 502, 504, 506 of step 206 can be performed with multiple candidate model ultrasound images substantially simultaneously, and the iterative BP algorithm can be performed by sequentially calculating the probability of accuracy of the different candidate model ultrasound images.

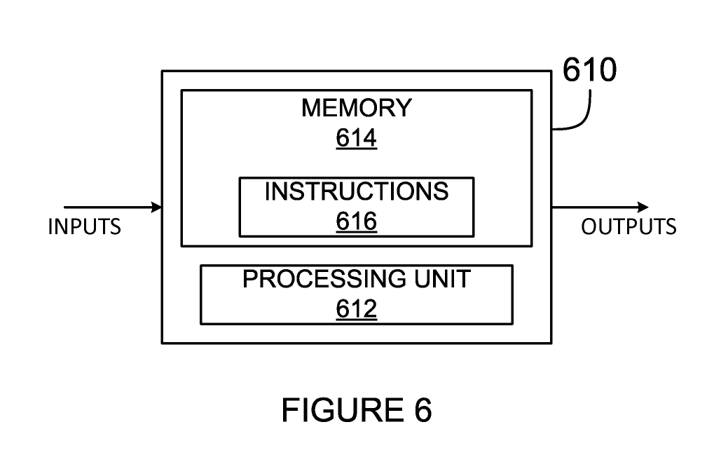

With reference to FIG. 6, the method 200 may be implemented by a computing device 610, comprising a processing unit 612 and a memory 614 which has stored therein computer-executable instructions 616. The processing unit 612 may comprise any suitable devices configured to cause a series of steps to be performed so as to implement the method 300 such that instructions 616, when executed by the computing device 610 or other programmable apparatus, may cause the functions/acts/steps specified in the methods described herein to be executed. The processing unit 612 may comprise, for example, any type of general-purpose microprocessor or microcontroller, a digital signal processing (DSP) processor, a central processing unit (CPU), an integrated circuit, a field programmable gate array (FPGA), a reconfigurable processor, other suitably programmed or programmable logic circuits, or any combination thereof.

The memory 614 may comprise any suitable known or other machine-readable storage medium. The memory 614 may comprise non-transitory computer readable storage medium such as, for example, but not limited to, an electronic, magnetic, optical, electromagnetic, infrared, or semiconductor system, apparatus, or device, or any suitable combination of the foregoing. The memory 614 may include a suitable combination of any type of computer memory that is located either internally or externally to device such as, for example, random-access memory (RAM), read-only memory (ROM), compact disc read-only memory (CDROM), electro-optical memory, magneto-optical memory, erasable programmable read-only memory (EPROM), and electrically-erasable programmable read-only memory (EEPROM), Ferroelectric RAM (FRAM) or the like. Memory may comprise any storage means (e.g., devices) suitable for retrievably storing machine-readable instructions executable by processing unit.

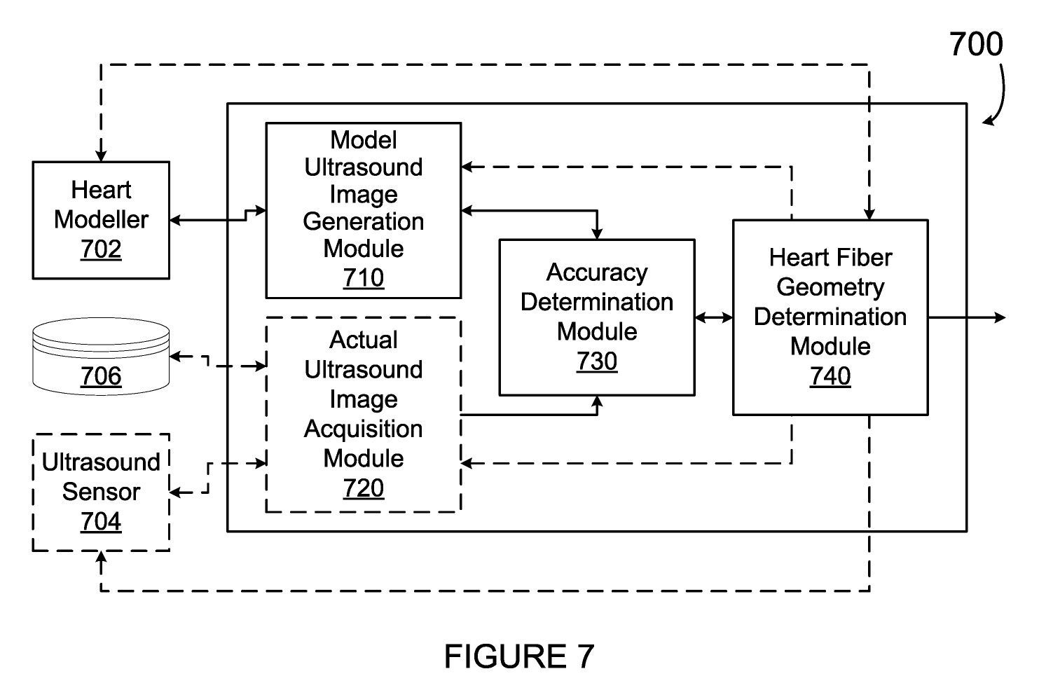

With reference with to FIG. 7, there is shown a system 700 for implementing the method 200 for determining heart fiber geometry for the heart 100. The system 700 comprises a model ultrasound image generation module 710, an accuracy determination module 730, a heart fiber geometry determination module 740, and optionally an actual ultrasound image acquisition module 720. The system 700 is communicatively coupled to a heart modeller 702, and optionally to one or more of an ultrasound sensor 704 and a database 706.

The model ultrasound image generation module 710 is configured for acquiring and/or generating model ultrasound images based on a heart model, in accordance with step 202. The heart model may be acquired from the heart modeller 702. In some embodiments, the model ultrasound image generation module 710 is configured for receiving a heart model from the heart modeller 702 and for generating the model ultrasound images. In some other embodiments, the model ultrasound image generation module 710 is configured for sending parameters for the model ultrasound images to the heart modeller 702 and for receiving the model ultrasound images from the heart modeller 702. In some embodiments, the model ultrasound image generation module 710 is communicatively coupled to the heart fiber geometry determination module 740 and receives instructions therefrom, for example to acquire and/or generate the model ultrasound images. The model ultrasound image generation module 710 is further configured for sending the model ultrasound images to the comparison unit 730.

Optionally, the system 700 includes the actual ultrasound image acquisition module 720. The actual ultrasound image acquisition module 720 is configured for acquiring the at least one actual ultrasound image, as per step 204, and for transmitting the actual ultrasound image to the accuracy determination module 730. The actual ultrasound image acquisition module 720 may acquire the actual ultrasound image from the ultrasound sensor 704 and/or from a database 706. In some embodiments, the actual ultrasound image acquisition module 720 controls the ultrasound sensor 704 and directs the ultrasound sensor 704 to acquire the actual ultrasound image. In some embodiments, the database 706 may be a local database, whereas in other embodiments the database 706 is remote and/or accessible via one or more networks, such as the Internet. In some embodiments, the actual ultrasound image acquisition module 720 is communicatively coupled to the heart fiber geometry determination unit and receives instructions therefor, for example to acquire the actual ultrasound image.

The accuracy determination module 730 is configured for receiving the model ultrasound image and the actual ultrasound image from the model ultrasound image generation module 710 and optionally from the actual ultrasound image acquisition module 720, respectively. Alternatively, the accuracy determination module 730 may obtain the actual ultrasound image directly from the database 706, or from some other source. The accuracy determination module 730 is further configured for determining a probability of accuracy of the model ultrasound images based at least in part on the actual ultrasound image, as per step 206.

The heart fiber geometry determination module 740 is configured for receiving the probability of accuracy from the accuracy determination module 730 and for deciding whether the probability of accuracy is above the given threshold, as per step 208. If the probability of accuracy is above the threshold, the heart fiber geometry determination module 740 is configured for applying the heart model to the actual ultrasound image(s), as per step 210. The heart fiber geometry determination module 740 may acquire the heart model from the heart modeller 720, or from any other suitable source.

The heart fiber geometry determination module 740 may be further configured for controlling the operation of the system 700, for example by issuing instructions to the model ultrasound image generation module 710, the actual ultrasound image acquisition module, the accuracy determination module 730, and one or more of the heart modeller 702 and the ultrasound sensor 704. For example, if the probability of accuracy is below the given threshold, the heart fiber geometry determination module 740 issues instructions to the accuracy determination module to calculate the probability of accuracy of a different candidate model ultrasound image. Alternatively, heart fiber geometry determination module 740 issues instructions to the ultrasound image generation module and/or the heart modeller 702 to generate new model ultrasound images. The heart fiber geometry determination module 740 may also be configured for instructing the actual ultrasound image acquisition module 720 and/or the ultrasound sensor 704 to acquire actual ultrasound images.

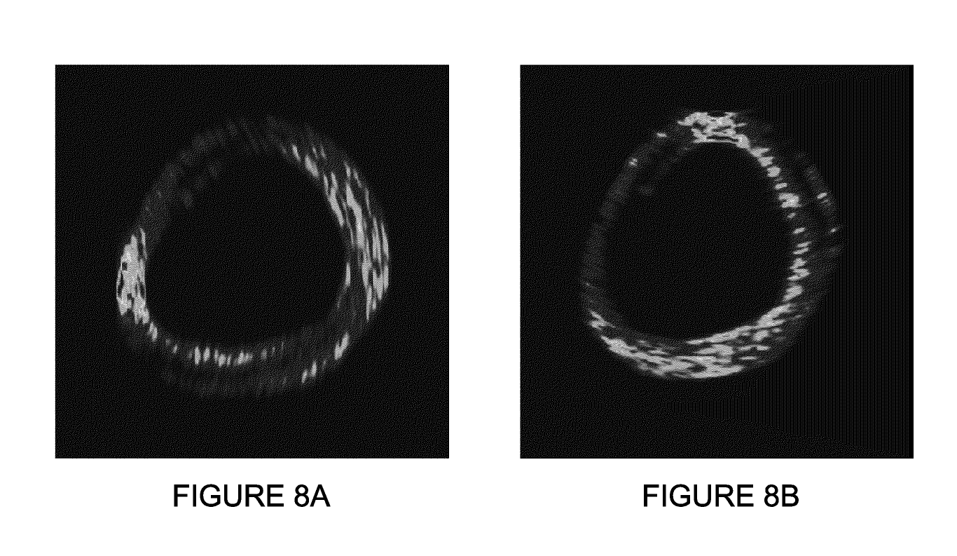

With reference to FIGS. 8A and 8B, there are shown two model ultrasound images as acquired by an embodiment of the model ultrasound image generation module 710 following an embodiment of step 202 of the method 200. FIG. 8A is acquired based on the simulated ultrasound beam being positioned at a “9 o'clock” position with respect to the heart model, and FIG. 8B is acquired based on the simulated ultrasound beam being positioned at a “12 o'clock” position with respect to the heart model. The model ultrasound images of FIGS. 8A-B are example B-mode USBS images. In this example, the model ultrasound images were generated using a modified version of the COLE ultrasound simulation software.

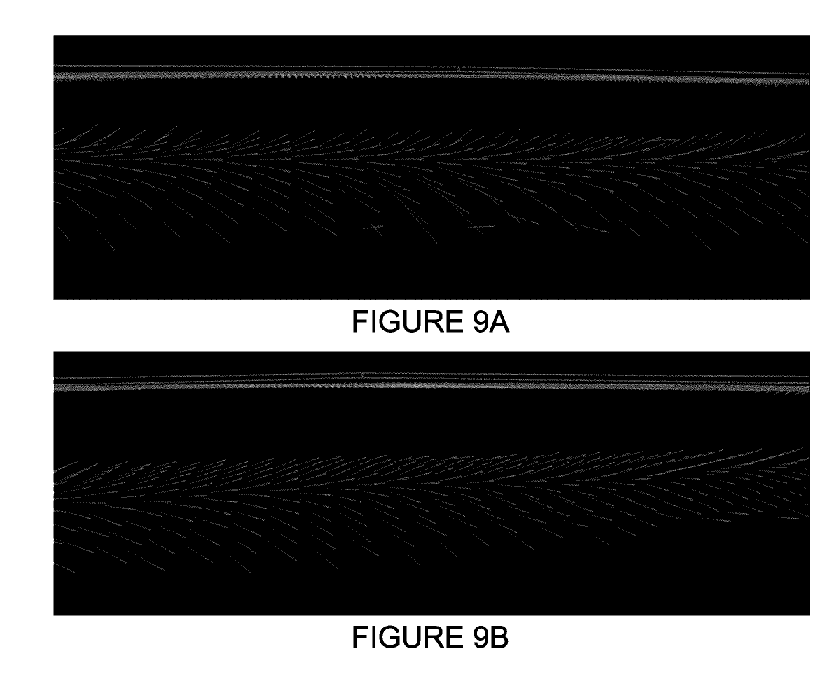

With reference to FIGS. 9A and 9B, there are shown two heart fiber geometries for the heart 100. FIG. 9A is a “ground truth” heart fiber geometry, and FIG. 9B is a heart fiber geometry as determined by an embodiment of the system 700 following an embodiment of the method 200. Specifically, in this example the prior model was a generalized helicoid model with six parameters per pixel: three determining the spatial orientation, and three determining the curvatures of the generalized helicoid model.

The unary potential was determined via a sum-of-squared-differences algorithm between the pixels of the model ultrasound images and the actual ultrasound images. The pairwise potential was determined via an average angular difference of overlap of two adjacent generalized helicoid model estimations. The BP algorithm used was a particle BP algorithm using 12 candidate solutions which were iteratively refined to obtain the heart fiber geometry of FIG. 9B.

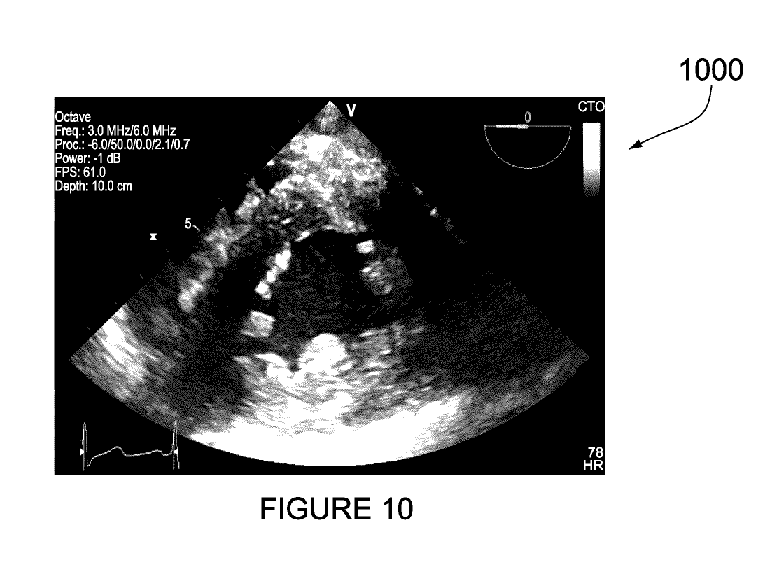

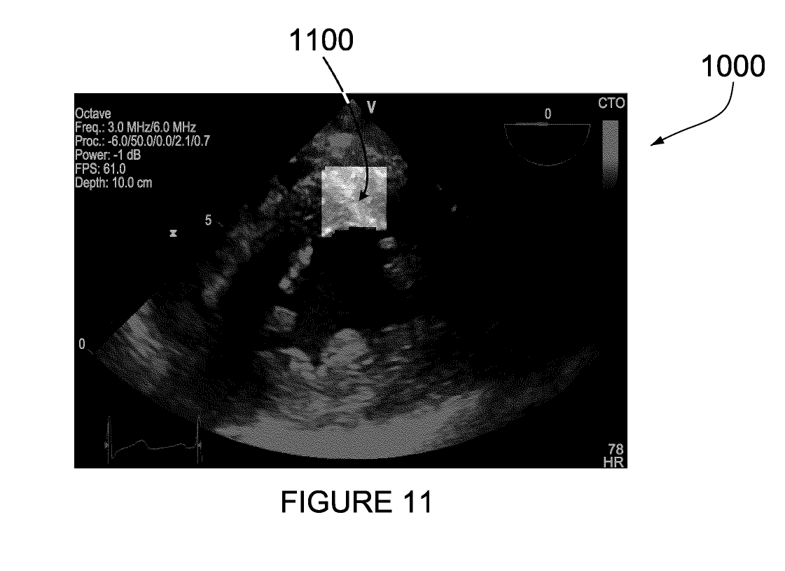

With reference to FIG. 10, the method 200 can be used in vivo to determine heart fiber geometry for a heart of an example patient. FIG. 10 illustrates a two-dimensional short-axis slice 1000 through an echocardiography acquisition from the example patient. The echocardiography acquisition can be acquired using any suitable means, for example by using ultrasound imaging techniques, including any of the ultrasound imaging techniques described hereinabove. The slice 1000 can be of any suitable portion of a heart of the patient. With reference to FIG. 11, a portion 1100 of the slice 1000, for example of a ventricular wall of the heart of the patient, can be used to illustrate an embodiment of the method 200.

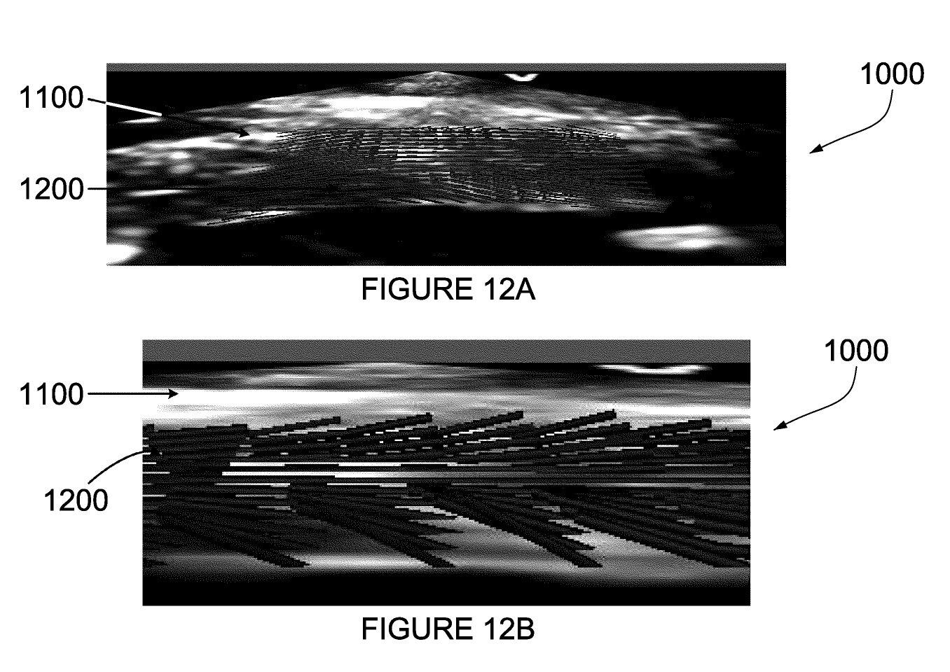

With reference to FIGS. 12A-B, one possible use of the method 200 is for inferring a fiber orientation field 1200 for the portion 1100 of the slice 1000. For example, the slice 1000 can be used as the ultrasound image of the heart acquired at optional step 204 and used to determine the probability of accuracy of the model ultrasound images at step 206. FIG. 12A is a wide-angle view of a fiber orientation field 1200 as determined by a particular embodiment of the method 200, and FIG. 12B is a zoomed-in view of the fiber orientation field 1200 where the viewing angle is almost coplanar with the plane of the slice 1000, in order to emphasize the variation of fiber orientation in the fiber orientation field 1200.

It should be noted that the fiber orientation field 1200 is one example of what can be done using the method 200, and that the fiber orientation field 1200 merely illustrates a result produced by a particular embodiment of the method 200. The method 200 can be used in different ways to produce different representations of determined heart fiber geometries.

The methods and systems for determining heart fiber geometry for a heart 100 described herein may be implemented in a high level procedural or object oriented programming or scripting language, or a combination thereof, to communicate with or assist in the operation of a computer system, for example the computing device 610. Alternatively, the methods and systems for determining heart fiber geometry for a heart 100 described herein may be implemented in assembly or machine language. The language may be a compiled or interpreted language. Program code for implementing the methods and systems for determining heart fiber geometry for a heart 100 described herein may be stored on a storage media or a device, for example a ROM, a magnetic disk, an optical disc, a flash drive, or any other suitable storage media or device. The program code may be readable by a general or special-purpose programmable computer for configuring and operating the computer when the storage media or device is read by the computer to perform the procedures described herein. Embodiments of the methods and systems for determining heart fiber geometry for a heart 100 described herein may also be considered to be implemented by way of a non-transitory computer-readable storage medium having a computer program stored thereon. The computer program may comprise computer-readable instructions which cause a computer, or more specifically the at least one processing unit of the computer, to operate in a specific and predefined manner to perform the functions described herein.

Computer-executable instructions may be in many forms, including program modules, executed by one or more computers or other devices. Generally, program modules include routines, programs, objects, components, data structures, etc., that perform particular tasks or implement particular abstract data types. Typically the functionality of the program modules may be combined or distributed as desired in various embodiments.

Various aspects of the methods and circuits for determining heart fiber geometry for a heart 100 disclosed herein may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments. Although particular embodiments have been shown and described, it will be obvious to those skilled in the art that changes and modifications may be made without departing from this invention in its broader aspects. The scope of the following claims should not be limited by the preferred embodiments set forth in the examples, but should be given the broadest reasonable interpretation consistent with the description as a whole.

1. A method for determining heart fiber geometry for a heart, comprising:
generating ultrasound images of a heart model;
determining a probability of accuracy of the ultrasound images of the heart model based at least in part on at least one ultrasound image of an actual heart; and
when the probability of accuracy is above a given threshold, applying the heart model to the at least one ultrasound image to determine heart fiber geometry of the actual heart.

2. The method of claim 1, wherein determining the probability of accuracy of the ultrasound images of the heart model comprises:
determining a similarity level indicative of a similarity between the ultrasound images of the heart model and the at least one ultrasound image of the actual heart;
determining a cohesion level indicative of a similarity between adjacent pixels of the ultrasound images of the heart model; and
computing the probability of accuracy of the ultrasound images of the heart model based on the similarity level and the cohesion level.

3. The method of claim 2, wherein determining a similarity level comprises applying a sum-of-squared differences algorithm.

4. The method of claim 2, wherein determining a cohesion level comprises applying an average angular difference algorithm.

5. The method of claim 1, wherein determining a probability of accuracy comprises applying a Bayesian interference belief propagation algorithm.

6. The method of claim 1, further comprising, when the probability of accuracy is below the given threshold:
creating new ultrasound images based on a new heart model; and
determining the probability of accuracy for the new ultrasound images.

7. The method of claim 1, wherein generating ultrasound images of a heart model comprises generating the ultrasound images of a mathematical heart model validated against an image dataset acquired using diffusion magnetic resonance imaging.

8. The method of claim 1, wherein generating ultrasound images of a heart model comprises generating the ultrasound images of the heart model from at least two different incoming ultrasound beam directions.

9. The method of claim 1, further comprising obtaining the at least one ultrasound image of the actual heart.

10. The method of claim 9, wherein obtaining the at least one ultrasound image of the actual heart comprises obtaining the at least one ultrasound image data set in vivo.

11. A system for determining heart fiber geometry for a heart, comprising:
a processing unit; and
a non-transitory memory communicatively coupled to the processing unit and comprising computer-readable program instructions executable by the processing unit for:
generating ultrasound images of a heart model;
determining a probability of accuracy of the ultrasound images of the heart model based at least in part on at least one ultrasound image of an actual heart; and
when the probability of accuracy is above a given threshold, applying the heart model to the at least one ultrasound image to determine heart fiber geometry of the actual heart.

12. The system of claim 11, wherein determining the probability of accuracy of the ultrasound images of the heart model comprises:
determining a similarity level indicative of a similarity between the ultrasound images of the heart model and the at least one ultrasound image of the actual heart;
determining a cohesion level indicative of a similarity between adjacent pixels of the ultrasound images of the heart model; and
computing the probability of accuracy of the ultrasound images of the heart model based on the similarity level and the cohesion level.

13. The system of claim 12, wherein determining a similarity level comprises applying a sum-of-squared differences algorithm.

14. The system of claim 12, wherein determining a cohesion level comprises applying an average angular difference algorithm.

15. The system of claim 11, wherein determining a probability of accuracy comprises applying a Bayesian interference belief propagation algorithm.

16. The system of claim 11, the computer-readable program instructions further executable for, when the probability of accuracy is below the given threshold:
creating new ultrasound images based on a new heart model; and
determining the probability of accuracy for the new ultrasound images.

17. The system of claim 11, wherein generating ultrasound images of a heart model comprises generating the ultrasound images of a mathematical heart model validated against an image dataset acquired using diffusion magnetic resonance imaging.

18. The system of claim 11, wherein generating ultrasound images of a heart model comprises generating the ultrasound images of the heart model from at least two different incoming ultrasound beam directions.

19. The system of claim 11, the computer-readable program instructions further executable for obtaining the at least one ultrasound image of the actual heart.

20. The system of claim 19, wherein obtaining the at least one ultrasound image of the actual heart comprises obtaining the at least one ultrasound image data set in vivo.
